(19) United States
(12) Patent Application Publication
(10) Pub. No.: US 2021/0196921 A1
(43) Pub. Date: Jul. 1, 2021

(54) ENERGY-CHANNEL FREQUENCY RESONANCE DEVICE

(71) Applicant: MYRIDIAN INTERNATIONAL CORP., Irvine, CA (US)

(72) Inventor: Russell S. LIN, Keelung City (TW)

(21) Appl. No.: 16/728,263

(22) Filed: Dec. 27, 2019

(51) Int. Cl. A61M 21/02; A61N 2/00; A61H 23/02; A61N 5/06

(52) U.S. Cl. CPC A61M 21/02; A61N 2/002; A61M 2021/0027; A61H 23/02; A61N 5/0618; A61N 2/004

(57) ABSTRACT

An energy-channel frequency resonance device can be selectively mounted in an apparatus, such as a chair, a shoulder and neck massager, a seat, or a mattress. A user can operate the apparatus to generate acoustic frequency, optical frequency, magnetic frequency and vibration frequency, to promote human body circulation for health effect.

### BACKGROUND OF THE INVENTION

### 1. Field of the Invention

The present invention relates to an energy-channel frequency resonance device, and more particularly to a resonance device integrating acoustic frequency, optical frequency, magnetic frequency and vibration frequency.

### 2. Description of the Related Art

Modern people suffer from poor sleep quality or insomnia because of stress, staying up late, or fatigue. It can be understood that stress, staying up late or fatigue can cause people to be in a bad condition, such as poor mental health, poor work efficiency, or dangerous driving.

In recent years, low-frequency magnetic stimulation signals, such as Schumann wave which is a kind of low-frequency magnetic field, are applied to human body, and it found that the low-frequency Schumann wave can form resonance with a user's brain, and the user's sleep quality and sleep efficiency can be improved.

At present, various colleges, universities and research centers have invested in relevant research, but inventors of the present invention find that low-frequency magnetic stimulation signal is not limited to the Schumann wave. When the relevant devices can be integrated to a device, such integrated device can be operated to make people fully rest and improve frequency of brain waves, and the inventors of the present invention believe that such integrated device can provide preventive health care effect for human body.

### SUMMARY OF THE INVENTION

An objective of the present invention is to provide an energy-channel frequency resonance device which can be selectively mounted in an apparatus such as a chair, a shoulder and neck massager, a seat, or a mattress, and when in use, the energy-channel frequency resonance device can generate acoustic frequency, optical frequency, magnetic frequency and vibration frequency, so as to promote human body circulation for health effect.

In order to achieve the objective, the present invention provides an energy-channel frequency resonance device basically including a Schumann wave generating unit, at least one resonating speaker, a vibrating unit, a light emitting unit and a control circuit.

The control circuit is electrically connected to the Schumann wave generating unit, the resonating speaker, the vibrating unit and the light emitting unit, and configured to generate and control various types of resonance modes.

The Schumann wave generating unit is configured to generate Schumann wave of 7.83 Hz. In an embodiment, the Schumann wave generating unit can use single chip to generate Schumann waveform with oscillating frequency, and drive a transistor to input the Schumann waveform to an amplifier circuit, so that the amplifier circuit can emit and transmit magnetic field of the Schumann wave to a human body.

The resonating speaker can broadcast music with specific frequency, and generate specific audio frequency having frequencies of twelve energy channels in Chinese medicine, so as to further generate low-frequency physical resonance, and then achieve bioresonance by sound vibration and physical vibration.

The vibrating unit can use a vibration motor to generate low-frequency vibration, so as to vibrate a portion of a human body, thereby promoting human body circulation.

The light emitting unit mainly generates various colors of lights to promote human body circulation. The energy-channel frequency resonance device of the present invention can generate bioresonance, and when the characteristic of the vibration generated by the energy-channel frequency resonance device is the same as that of a human body, resonance can be generated, so that brain wave can be adjusted from dysphoria beta (β) wave to relaxing and soothing alpha (α) wave, thereby making frequency of the human body harmonious.

In an embodiment of the present invention, the control circuit can include a transmission unit and a controller, and the energy-channel frequency resonance device can generate bioresonance through the transmission unit, by a wired control manner.

In an embodiment of the present invention, the control circuit includes a wireless transmission unit, and the energy-channel frequency resonance device can generate bioresonance through wireless transmission unit, by a wireless control manner.

### BRIEF DESCRIPTION OF THE DRAWINGS

The structure, operating principle and effects of the present invention will be described in detail by way of various embodiments which are illustrated in the accompanying drawings.

FIG. 1 is a system framework diagram of an energy-channel frequency resonance device of the present invention.

FIG. 2 is a schematic view of a first embodiment of an energy-channel frequency resonance device of the present invention.

FIG. 3 is a schematic view of a second embodiment of an energy-channel frequency resonance device of the present invention.

FIG. 4 is a schematic view of a third embodiment of an energy-channel frequency resonance device of the present invention.

FIG. 5 is a schematic view of a fourth embodiment of an energy-channel frequency resonance device of the present invention.

### DETAILED DESCRIPTION OF THE PREFERRED EMBODIMENTS

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

It is to be acknowledged that, although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the word “comprise”, “include” and “have”, and variations such as “comprises”, “comprising”, “includes”, “including”, “has” and “having”, will be acknowledged to imply the inclusion of stated elements but not the exclusion of any other elements.

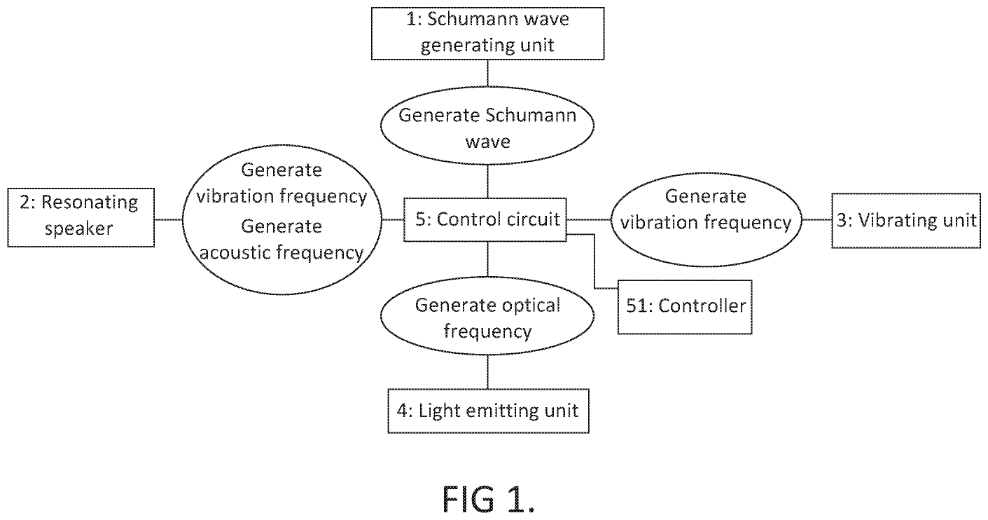

Please refer to FIG. 1, which is a schematic system diagram of an energy-channel frequency resonance device of the present invention. As shown in FIG. 1, the energy-channel frequency resonance device mainly includes a Schumann wave generating unit 1 and at least one resonating speaker 2, and a vibrating unit 3, a light emitting unit 4 and a control circuit 5.

The Schumann wave generating unit 1 is configured to generates magnetic frequency resonance; for example, the Schumann wave generating unit 1 can generate Schumann wave of 7.83 Hz. In an embodiment, the Schumann wave generating unit 1 can use single chip to generate a Schumann waveform with oscillating frequency, and drive a transistor to input the Schumann waveform to an amplifier circuit, so that the amplifier circuit emits and transmits magnetic field of the Schumann wave to human body.

The resonating speaker 2 is configured to generate two bioresonance effects including an acoustic frequency resonance effect and a vibration frequency resonance effect. The resonating speaker 2 can broadcast music and generate specific frequencies of twelve energy channels in Chinese medicine, so as to generate low-frequency resonance to promote human body circulation.

The vibrating unit 3 can use a vibration motor to generate low-frequency physical vibration to vibrate a portion of a human body, so as to promote body metabolism. Since the neural signal conduction and adjustment of the human body are performed by vibration, when a different vibration source appears inside the human body, the abnormal vibration may make a user feel uncomfortable, for example, the user may have neck and shoulder soreness, or lower back soreness. The vibrating unit 3 can be used to change the vibration effect inside human body.

The light emitting unit 4 is mainly used to generate various colors of lights to promote human body circulation. For example, the light emitting unit 4 can generate an infrared light, which is warm to promote blood circulation; a blue light, which is a kind of cold light; a green light, which can reduce inflammation, stop swelling and balance body frequency; an orange light, which is helpful for digestive organ; a purple light, which has effect of Yin and Yang complementary, and can enrich essence and supplement marrow; or, the light emitting unit 4 can generate various colors of lights to achieve color-light resonance effect. In an embodiment of the present invention, the infrared light is taken as an example, the light emitting unit 4 has an infrared film which is disposed thereon and made by nano powder with 10% far infrared material, so that the light emitting unit 4 can be used to promote human body circulation by light and the medium containing the far infrared nano material.

The control circuit 5 is electrically connected to the Schumann wave generating unit 1, the resonating speaker 2, the vibrating unit 3 and the light emitting unit 4. The various types of resonance modes can be generated under the control of the control circuit 5. As shown in FIG. 1, in order to enable the Schumann wave generating unit 1, the resonating speaker 2, the vibrating unit 3 and the light emitting unit 4 to operate independently, operate cooperatively, or operate a local function only, the control circuit 5 can include a controller 51 configured to issue a turn-on/off instruction to each of the above-mentioned units/devices through a transmission unit. In an embodiment, the control circuit 5 can include a wireless transmission unit configured to use a wireless communication technology, such as Bluetooth, WiFi, Zbee, infrared remote control manner, to enable a smart device (such as a mobile phone, a tablet computer, a computer, or a remote controller) of a user to send a control instruction to each of above-mentioned units/devices.

As shown in FIG. 1, the energy-channel frequency resonance device of the present invention can generate bioresonance such as acoustic frequency, vibration frequency, optical frequency, Schumann wave with low magnetic frequency, and so on. When the characteristic of the vibration generated by the energy-channel frequency resonance device is the same as that of human body, resonance can be generated, so that brain wave can be adjusted from dysphoria beta (β) wave to relaxing and soothing alpha (α) wave, thereby making the frequency of the human body harmonious.

Furthermore, the control circuit the of the present invention can generate bioresonance in different modes corresponding to different portions of human body. For the acoustic frequency resonance, a human body has twelve energy channels including a liver channel, a kidney channel, a spleen pancreas channel, a lung channel, a stomach channel, a gall bladder channel, a bladder channel, a large intestine channel, a triple heatera channel, a small intestine channel, a heart channel, and a pericardium channel. During operation, the user can control the controller 51 or the smart device to select the corresponding mode to perform bioresonance. Table 1 lists frequencies of the twelve energy channels.

TABLE 1
listing frequencies of twelve energy channels

| Frequency | Title of energy channel |
|---|---|
| 1. Beat(1.2 HZ) | Liver channel |
| 2. Beat(2.4 HZ) | Kidney channel |
| 3. Beat(3.6 HZ) | Spleen Pancreas |
| 4. Beat(4.8 HZ) | Lung channel |
| 5. Beat(6.0 HZ) | Stomach channel |
| 6. Beat(7.2 HZ) | Stomach channel |
| 7. Beat(8.4 HZ) | Bladder channel |
| 8. Beat(9.6 HZ) | Large Intestine channel |
| 9. Beat(10.8 HZ) | Triple Heater channel |
| 10. Beat(12.0 HZ) | Small Intestine channel |
| 11. Beat(13.2 HZ) | Heart channel |
| 12. Beat(14.4 HZ) | Pericardium channel |

Besides the bioresonance resonance modes for the twelve energy channels by acoustic frequency, the energy-channel frequency resonance device of the present invention can integrate acoustic frequency, optical frequency, magnetic frequency and vibration frequency to generate the bioresonance modes for nine constitutions including a qi-deficiency constitution, a qi-depression constitution, a blood-stasis constitution, a yin-deficiency constitution, a yang-deficiency constitution, a damp-heat constitution, a phlegm-dampness constitution, a specific-endowment constitution, and a mild constitution. The appropriate resonance mode can be selected according to a condition of different user, to promote human body metabolism and circulation. Table 2 lists biological characteristics of the nine constitutions.

TABLE 2
listing biological characteristics of nine constitutions

| Title | Characteristic | Title | Characteristic |
|---|---|---|---|
| Qi-deficiency | Palpitations, | Damp-heat | Sore, heat, damp |
| constitution | discouragement, | constitution | |
| | sweating | | |
| Qi depression | Depression, anxiety, | Phlegm-dampness | Obesity, phlegm, |
| constitution | weak | constitution | damp |
| Blood-stasis | Dim skin, speckle, pain | Specific endowment | Allergy |
| constitution | | constitution | |
| Yin deficiency | Dry mouth, heat, thin | Mild constitution | Harmonious |
| constitution | | | balance |
| Yang deficiency | Chilling, Impotence, | | |
| constitution | low activity | | |

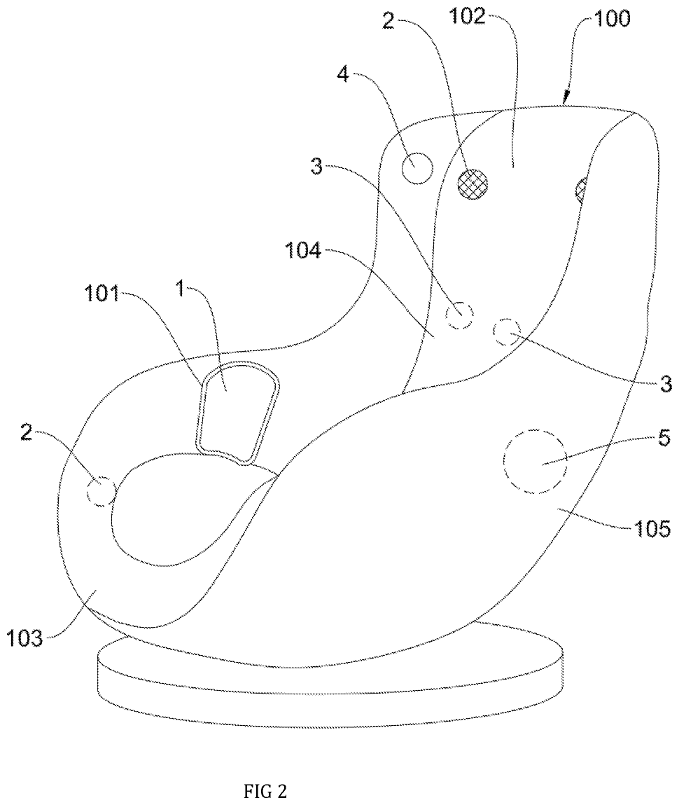

Please refer to FIG. 2, which shows a first embodiment of the present invention. In the first embodiment, the energy-channel frequency resonance device is applied to a massage chair 100; preferably, a user can lie on the massage chair 100 for massaging, so that gravity can be shared to many portions of the body with a lying posture, thereby preventing only some portions of the body from bearing pressure. As shown in FIG. 2, the Schumann wave generating unit 1 is disposed on an armrest portion 101 of the massage chair 100, a resonating speaker 2 is disposed on a headrest portion 102 and a lower-leg portion 103 of the massage chair 100, the vibrating unit 3 is disposed on a lower back portion 104 of the massage chair 100, and the light emitting unit 4 is disposed aside the headrest portion 102. Circuits of the Schumann wave generating unit 1, the resonating speaker 2, the vibrating unit 3 and the light emitting unit 4 can be arranged and integrated in the control circuit 5 disposed at the rear end 105. The user can operate the controller 51 or a smart device to select one of the above-mentioned modes, so as to start massage and bioresonance.

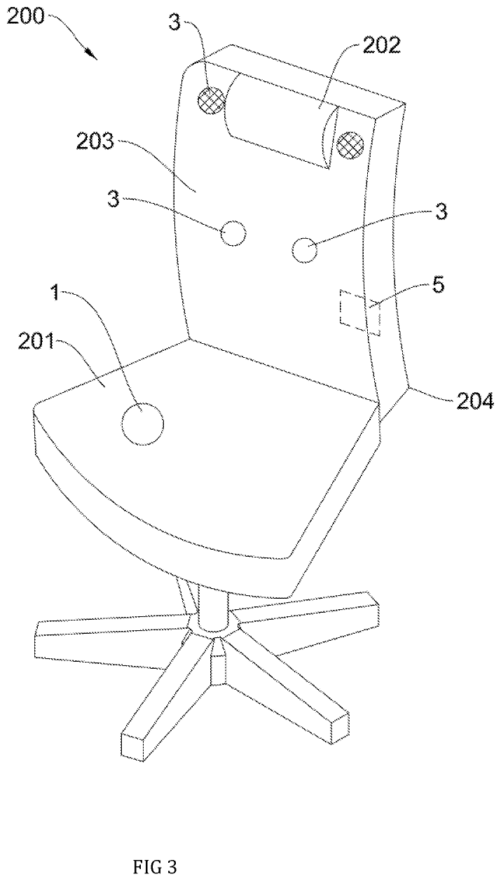

Please refer to FIG. 3, which shows a second embodiment of the present invention. In the second embodiment, the energy-channel frequency resonance device of the present invention can be applied to an ergonomic chair 200; preferably, a user can work and accept resonance status on the ergonomic chair 200. In this embodiment, in order to make the user focus on work, the energy-channel frequency resonance device is not provided with the light emitting unit 4. As shown in FIG. 3, the Schumann wave generating unit 1 is disposed on a seat portion 201 of the ergonomic chair 200, the resonating speaker 2 is disposed on a headrest portion 202 of the ergonomic chair 200, and the vibrating unit 3 is disposed in a lower back portion 203 of the ergonomic chair 200. Circuits of the Schumann wave generating unit 1, the resonating speaker 2 and the vibrating unit 3 can be arranged and integrated in the control circuit 5 at a rear end 204 of the ergonomic chair 200. The user can operate the controller 51 or a smart device to select one of the above-mentioned modes, to perform bioresonance.

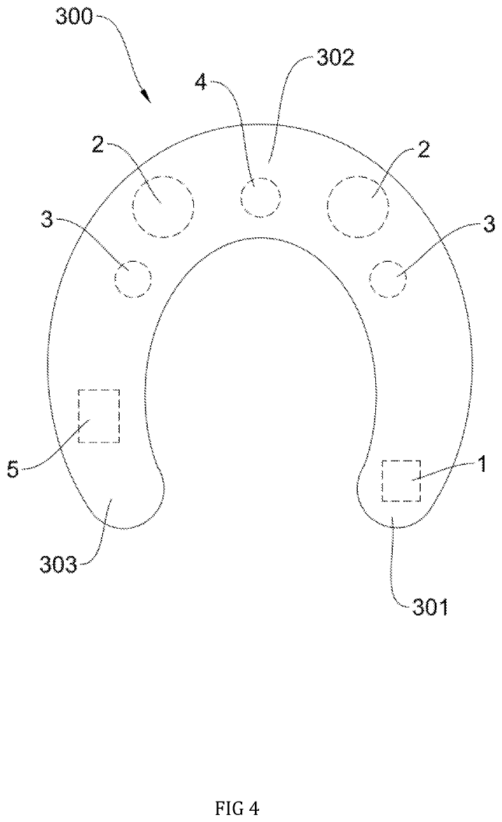

Please refer to FIG. 4, which shows a third embodiment of the present invention. In the third embodiment, the energy-channel frequency resonance device of the present invention is applied to a shoulder and neck massager 300. As shown in FIG. 4, the Schumann wave generating unit 1 is disposed on a first end 301 of the shoulder and neck massager 300; the resonating speaker 2, the vibrating unit 3 and the light emitting unit 4 are disposed in a middle section 302 of the shoulder and neck massager 300. Circuits of the Schumann wave generating unit 1, the resonating speaker 2, the vibrating unit 3 and the light emitting unit 4 can be arranged and integrated in the control circuit 5 at a second end 303 of the shoulder and neck massager 300. The user can operate the controller 51 or the smart device to select one of the above-mentioned modes, to perform bioresonance.

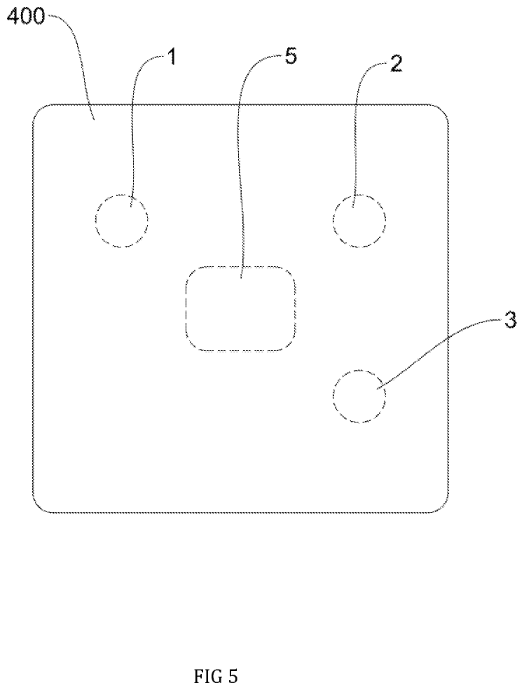

Please refer to FIG. 5, which shows a fourth embodiment of the present invention. In the fourth embodiment, the energy-channel frequency resonance device of the present invention is applied to a seat cushion 400. The Schumann wave generating unit 1, the resonating speaker 2 and the vibrating unit 3 are appropriately disposed in the seat cushion 400, and circuits of the Schumann wave generating unit 1, the resonating speaker 2 and the vibrating unit 3 can be arranged and integrated in the control circuit 5. The user can operate the controller 51 or a smart device to select one of the above-mentioned modes, to perform bioresonance.

Besides the embodiments shown in FIGS. 2 to 5, the energy-channel frequency resonance device of the present invention can also be applied to an apparatus (such as a mattress, a car seat and so on) related to lying or sitting posture, so as to vibrate portions of human body and adjust frequencies of energy channels in Chinese medicine, thereby making seven chakras harmonious.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. An energy-channel frequency resonance device, comprising:
a Schumann wave generating unit configured to generate a Schumann wave of 7.83 Hz, wherein the Schumann wave generating unit uses at least one single chip to generate a Schumann waveform with oscillating frequency, and drives a transistor to input the Schumann waveform to an amplifier circuit, so that the amplifier circuit emits and transmits magnetic field of the Schumann wave to a human body;
at least one resonating speaker configured to broadcast music, and generate specific frequencies of twelve energy channels in Chinese medicine, so as to generate low-frequency physical resonance, thereby achieving bioresonance through sound vibration or physical vibration,
a vibrating unit configured to use a vibration motor to generate low-frequency vibration;
a light emitting unit configured to generate various colors of lights to promote human body circulation; and
a control circuit electrically connected to the Schumann wave generating unit, the resonating speaker, the vibrating unit and the light emitting unit, and configured to control and generate various types of resonance modes.

2. The energy-channel frequency resonance device according to claim 1, wherein the control circuit comprises a transmission unit and a controller, and the controller controls the energy-channel frequency resonance device through the transmission unit, by a wired control manner.

3. The energy-channel frequency resonance device according to claim 1, wherein the control circuit comprises a wireless transmission unit, and the control circuit controls the energy-channel frequency resonance device through the wireless transmission unit, by a wireless control manner.

4. The energy-channel frequency resonance device according to claim 1, wherein the control circuit comprises resonance modes for resonating the twelve energy channels.

5. The energy-channel frequency resonance device according to claim 1, wherein the control circuit comprises bioresonance modes for nine constitutions.

6. The energy-channel frequency resonance device according to claim 1, wherein the energy-channel frequency resonance device is applied to a massage chair, the Schumann wave generating unit is disposed on an armrest portion of the massage chair, the resonating speaker is disposed on a headrest portion and a lower-leg portion of the massage chair, the vibrating unit is disposed on a lower back portion of the massage chair, the light emitting unit is disposed aside the headrest portion, and circuits of the Schumann wave generating unit, the resonating speaker, the vibrating unit and the light emitting unit are arranged and integrated in the control circuit in a rear end of the massage chair.

7. The energy-channel frequency resonance device according to claim 1, wherein the energy-channel frequency resonance device is applied to an ergonomic chair, the Schumann wave generating unit is disposed on a seat portion of the ergonomic chair, the resonating speaker is disposed on a headrest portion of the ergonomic chair, the vibrating unit is disposed in a lower back portion of the ergonomic chair, and circuits of the Schumann wave generating unit, the resonating speaker and the vibrating unit are arranged and integrated in the control circuit at a rear end of the ergonomic chair.

8. The energy-channel frequency resonance device according to claim 1, wherein the energy-channel frequency resonance device is applied to a shoulder and neck massager, the Schumann wave generating unit is disposed on a first end of the shoulder and neck massager, wherein the resonating speaker, the vibrating unit and the light emitting unit are disposed in a middle section of the shoulder and neck massager, and circuits of the Schumann wave generating unit, the resonating speaker, the vibrating unit and the light emitting unit are arranged and integrated in the control circuit at a second end of the shoulder and neck massager.
